(19) United States
(12) Patent Application Publication
(10) Pub. No.: US 2010/0298644 A1
(43) Pub. Date: Nov. 25, 2010

(54) CO-AXIAL ORAL INTUBATION DEVICE AND SYSTEM

(76) Inventor: Bruce Kleene, Rochester, NY (US)

Correspondence Address: HESLIN ROTHENBERG FARLEY & MESITI PC, 5 COLUMBIA CIRCLE, ALBANY, NY, 12203, US

(21) Appl. No.: 12/468,430

(22) Filed: May 19, 2009

(51) Int. Cl. A61B 1/267; A61M 16/04

(52) U.S. Cl. 600/188; 600/194; 128/207.14

(57) ABSTRACT

A co-axial oral intubation device includes a generally J-shaped blade (flat or curved) with handle portion to deliver a flexible airway instrument, such as a fiber optic bronchoscope, when coupled to the blade into the trachea of a patient for subsequent co-axial intubation with the flexible airway instrument. The blade is long enough that the handle portion remains outside the patient for manipulation, after insertion into the patient's airway. An enclosed guide is coupled to the back side of the blade, conforming to the blade, for guiding and holding the flexible airway instrument. An oral intubation system includes the co-axial oral intubation device, together with a flexible airway instrument.

### BACKGROUND OF THE INVENTION

1. Technical Field

The present invention generally relates to tracheal intubation. More particularly, the present invention relates to a device for assisting with delivering a flexible airway instrument into the trachea of a patient, the flexible airway instrument used to co-axially deliver an endotracheal tube.

2. Background Information

Tracheal intubation, the insertion of an endotracheal tube into the trachea, is a common medical procedure. Tracheal intubation is done in the initial stage of a general anesthetic and at other times in order to permit mechanical ventilation of the lungs, protection of the airway, and for other reasons. It is performed by anesthesiologists, nurse anesthetists, respiratory therapists, ED physicians, Emergency Medical Technicians, and by other health care workers.

The fiber optic bronchoscope is one example of a flexible airway instrument used for tracheal intubation. It has a flexible shaft of small diameter containing fiberoptic bundles for illumination and for viewing. A proximal hand piece contains an eyepiece and control elements. There is a light source contained within the hand piece or connected to it. The operator looks into the eyepiece and “sees” out the distal end of the shaft. Alternatively, the image may be displayed on a screen. In another example, a small videoscopic camera is placed at the end of a flexible shaft. In the present application, for the sake of simplicity, reference to a fiber optic bronchoscope is intended to refer also to the videoscopic instrument.

Tracheal intubation with a fiber optic bronchoscope is typically accomplished as follows. The operator inserts the distal end of the fiber optic bronchoscope through the oropharynx and into the hypopharynx of the patient to acquire an image of the vocal cords. He or she then advances the distal end of the fiber optic bronchoscope between the vocal cords and into the trachea. The operator then slides an endotracheal tube, which has been pre-loaded onto the proximal shaft of the instrument, distally over the fiber optic bronchoscope and into the trachea. The fiber optic bronchoscope is then removed, leaving the endotracheal tube in place.

Intubation with a fiber optic bronchoscope has several advantages over intubation with a traditional laryngoscope. The fiber optic bronchoscope depends less upon manipulation of the positions of the head, neck and mouth. Such manipulations may not be possible, due to anatomic constraints or contraindications such as known or suspected cervical spine injury. The fiber optic bronchoscope is also better adapted to intubation in the presence of some supraglottic or pharyngeal masses. Under such circumstances, intubation may be possible with a fiber optic bronchoscope, but impossible with traditional direct laryngoscopy.

Another advantage of a bronchoscope over the use of a laryngoscope is that intubation with a fiber optic bronchoscope is coaxial. The path of sight (along the shaft of fiber optic bronchoscope) and the path to intubation (also along the shaft of the fiber optic bronchoscope) are the same. Once the fiber optic bronchoscope is in proper position, passage of the endotracheal tube is generally readily accomplished. However, this is not necessarily so with traditional laryngoscopy. In traditional laryngoscopy, the endotracheal tube is passed into the hypopharynx alongside the laryngoscope, and from there into the trachea. Anatomic constraints may make it difficult to place the endotracheal tube even when the vocal cords are in view.

In light of these advantages, a fiber optic bronchoscope is used when certain anatomic conditions are present, when a difficult intubation is expected, or when other approaches have led to failed intubation attempts.

Nonetheless, there are several factors that limit the usefulness of the fiber optic bronchoscope. A view of the vocal cords may be difficult to acquire through the fiber optic bronchoscope. Soft tissues, such as the tongue, tonsils, and abnormal masses, may obstruct the view of the vocal cords and interfere with attempts to direct the distal end of the fiber optic bronchoscope. Fluids, such as blood and saliva, may interfere with the ability of the operator to see. Since most intubations are performed with the patient in the supine position, gravity causes secretions to pool in the posterior pharynx. Gravity also causes the path of the fiber optic bronchoscope to be along the posterior pharynx, increasing the likelihood that the secretions will obscure the distal end of the fiber optic bronchoscope, making the vocal cords difficult or impossible to see.

In addition, it is difficult to become skillful in the use of a fiber optic bronchoscope or other flexible airway instrument and to maintain that skill. The modern fiber optic bronchoscope is a sophisticated instrument not frequently in use by those who perform endotracheal intubations. Most anesthesia practitioners have been trained in its use but do not use it enough to maintain a high level of skill.

Thus, a need exists for a less complex way to achieve the co-axial insertion of the distal end of a fiber optic bronchoscope or other flexible airway instrument into the trachea of a patient.

### SUMMARY OF THE INVENTION

Briefly, the present invention satisfies the need for a less complex way to enter the trachea with a flexible airway instrument by providing a co-axial oral intubation device that is simpler in design and easier to manipulate than a fiber optic bronchoscope, for example.

In accordance with the above, it is an object of the present invention to provide a simple, easy-to-use device for facilitating co-axial intubation with a flexible airway instrument.

The present invention provides, in a first aspect, a co-axial oral intubation device. The device comprises a generally J-shaped blade with handle portion to deliver a flexible airway instrument when coupled to the blade into the trachea of a patient for subsequent co-axial intubation with the flexible airway instrument, the blade being long enough that the handle portion remains substantially outside the patient after placement in the patient. The device further comprises an enclosed guide coupled to a back side of the blade for guiding and holding the flexible airway instrument when present, the guide conforming to the blade.

The present invention provides, in a second aspect, a system for oral intubation. The system comprises a flexible airway instrument, and a co-axial oral intubation device. The device comprises a generally J-shaped flat blade with handle portion to deliver the flexible airway instrument when coupled to the blade into a trachea of a patient for subsequent co-axial intubation with the flexible airway instrument, the blade being long enough that the handle portion remains substantially outside the patient after placement in the patient. The device also comprises an enclosed guide coupled to a back side of the blade for guiding and holding the flexible airway instrument, the guide conforming to the blade.

These, and other objects, features and advantages of this invention will become apparent from the following detailed description of the various aspects of the invention taken in conjunction with the accompanying drawings.

### BRIEF DESCRIPTION OF THE DRAWINGS

FIG. 1 depicts one example of a system for oral intubation in accordance with the present invention.

FIG. 2 depicts the handle portion of the blade remaining substantially outside a patient after placement in accordance with the present invention.

FIG. 3 depicts one example of a co-axial oral intubation device in accordance with an aspect of the present invention.

FIG. 4 depicts the device of FIG. 3 after tightening the length of suture or tape.

FIG. 5 depicts another example of a co-axial oral intubation device in accordance with another aspect of the present invention.

FIG. 6 depicts the device of FIG. 5 after coupling the enclosed guide to the blade.

FIG. 7 depicts another example of a co-axial oral intubation device in accordance with another aspect of the present invention.

FIG. 8 is a close-up view of the distal end of the device of FIG. 7.

FIG. 9 depicts another example of a co-axial oral intubation device in accordance with another aspect of the present invention.

FIG. 10 depicts still another example of a co-axial oral intubation device in accordance with another aspect of with the present invention.

FIG. 11 depicts the device of FIG. 10 after tightening the length of the suture or tape.

FIG. 12 depicts another example of a co-axial oral intubation device in accordance with another aspect of the present invention.

FIG. 13 depicts the device of FIG. 12 after coupling the enclosed guide and blade via the post-and-hole connection.

FIG. 14 depicts yet another example of a co-axial oral intubation device in accordance with another aspect of the present invention.

FIG. 15 depicts the device of FIG. 14 with optional endotracheal or similar tube.

FIG. 16 depicts another example of a co-axial oral intubation device in accordance with another aspect of the present invention.

FIGS. 17(a) and 17(b) depict portions of the device of FIG. 16.

FIG. 18 depicts another example of a co-axial oral intubation device in accordance with another aspect of the present invention.

FIG. 19 depicts the device of FIG. 18 after bringing the secondary blade into contact with the primary blade.

FIG. 20 is a close-up view of the distal end of the device of FIG. 18 with optional endotracheal or similar tube.

FIG. 21 depicts still another example of a co-axial oral intubation device in accordance with another aspect of the present invention.

FIG. 22 depicts the device of FIG. 21 with the fiber-optic bronchoscope partially advanced.

FIG. 23 depicts one example of a generally J-shaped blade according to an aspect of the present invention.

FIG. 24 depicts one example of a malleable blade according to another aspect of the present invention.

### DETAILED DESCRIPTION OF THE INVENTION

The Co-axial Oral Intubation Device (COID) of the present invention facilitates use of a fiber optic bronchoscope or other flexible airway instrument for co-axial tracheal intubation by making it easier for the operator to insert the distal end of the fiber optic bronchoscope into the trachea. The COID makes the insertion easier by facilitating the acquisition of an image of the vocal cords, as described more fully below.

In all of the examples below, the blade of the COID may be made of any suitable hard material (unless otherwise specified), such as, for example, plastic or metal. In addition, unless otherwise specified, when in use with a flexible airway instrument for intubation, the COID may be released, leaving the flexible airway instrument, by sliding it back along the proximal length of the flexible airway instrument.

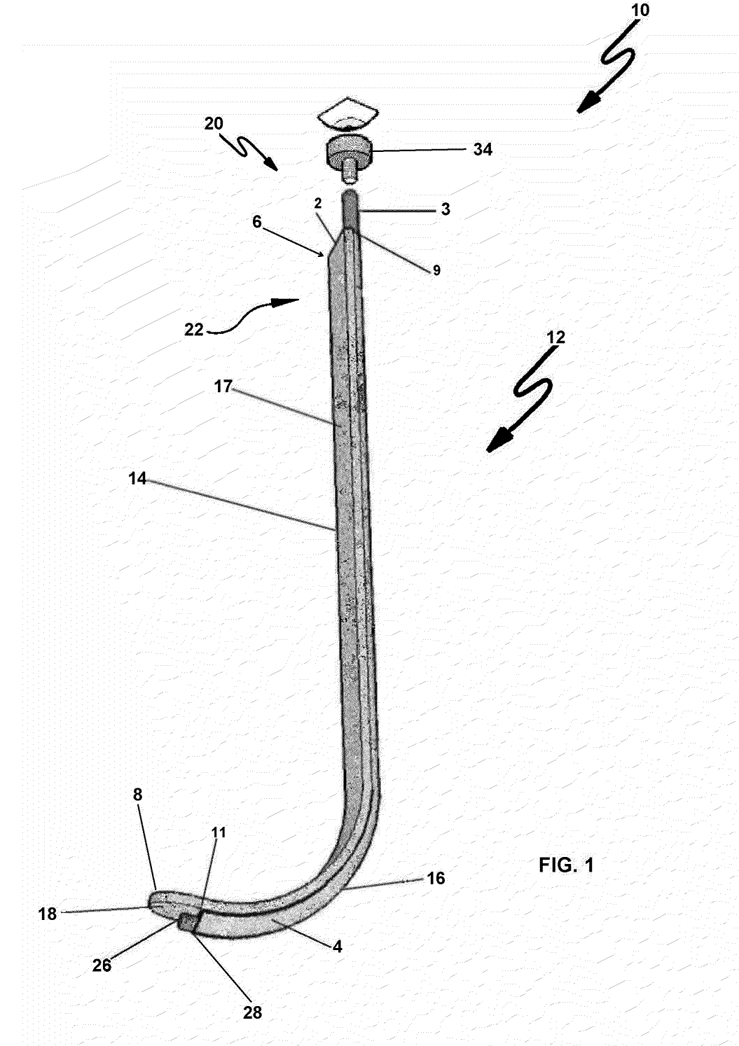

FIG. 1 depicts one example of a system 10 for oral intubation according to an aspect of the present invention. The system comprises a co-axial oral intubation device (COID) 12, comprising a blade 14 and an enclosed guide 16 coupled to a back side 18 of the blade and conforming therewith. The blade has a top 2, a bottom 4, and proximal 6 and distal 8 ends. It is generally J-shaped along its length, with the upstroke of the “J” at the distal end and in the direction of the top. The blade may be flat along its width or may be arcuate, with the concavity on the back. It may be constructed entirely of metal or of molded plastic, or it may be constructed of combinations of materials.

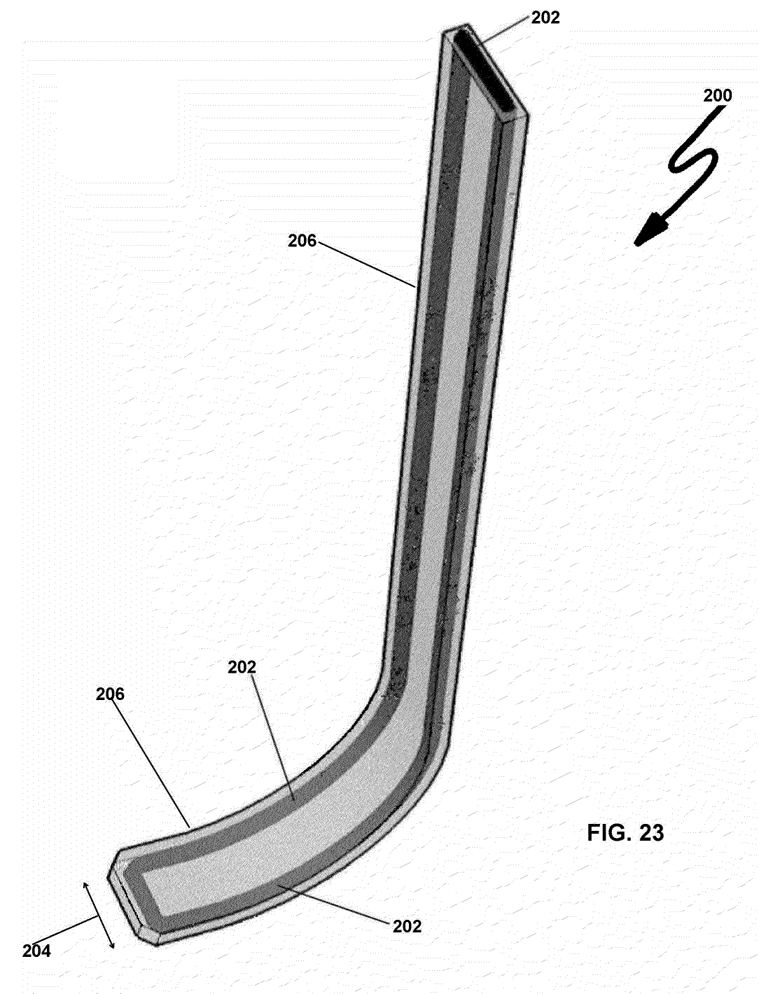

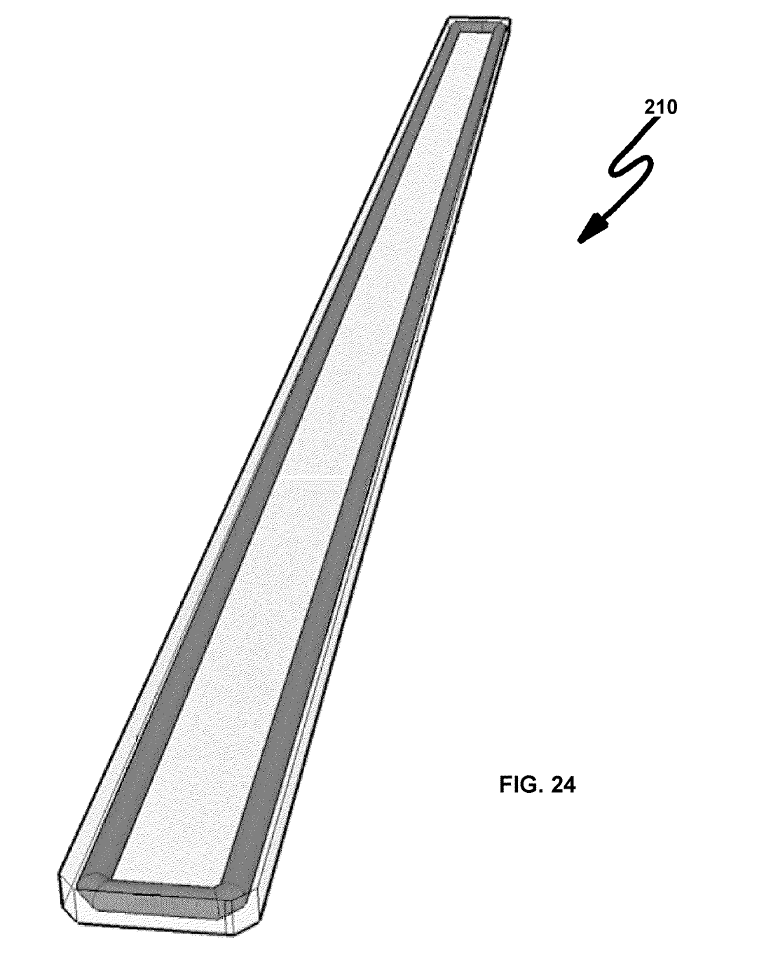

In one example, depicted in FIG. 23, the blade 200 comprises a metal element 202 such as a length of wire or metal bar about 1 to 2 mm thick and about 1 to 2 cms apart 204 from each other about a periphery of the blade. The metal element is covered in plastic 206. The plastic may be a sheath of thin plastic less than about 0.5 mm thick, or may be molded soft or hard plastic, within which the metal element is embedded. The plastic conforms to the shape of the metal element and forms the inner part of the blade by “connecting” the metal elements. Alternatively, a flexible sheath of polyester mesh or fabric or of another medical fabric may be used instead of plastic to cover the blade. The blade may be rigid or malleable. If malleable, it is preferably shapeable by the operator and yet rigid enough to retain the operator-determined shape against the typical forces arising during endotracheal intubation. The malleability gives the operator the capacity to adapt the blade to the requirements of the clinical situation, either immediately before attempting intubation or after one or more attempts. One example of a malleable blade is constructed of a stainless steel bar about 1.5 mm thick embedded within molded, soft plastic, as described above. Another example is a flat blade of stainless steel about 1 mm thick and 1 cm wide. Malleable blades would preferentially be manufactured and supplied to the end-user/operator in an unbent form. FIG. 24 depicts a malleable version 210 of the blade depicted in FIG. 23, as it would be preferably supplied to the operator. The operator would then bend the blade to produce a instrument of desired shape, yielding a blade similar in shape to that depicted in FIG. 23.

Returning to FIG. 1, the enclosed guide 16 provides for guiding and holding of the bronchoscope 20. The enclosed guide in this example takes the form of a sheath connected to the back of the blade. The sheath may be formed out of plastic, polyester mesh or fabric or another appropriate medical fabric. The enclosed guide has a top 9, a bottom 11, and proximal and distal ends corresponding to those of the blade. The guide is coupled to the blade in such a way that its top conforms to the bottom of the blade. The enclosed guide may itself be a tubular or sheath-like entity, open at the proximal and distal ends. In this case, the enclosed guide by itself comprises the conduit for the passage of the flexible airway instrument. The enclosed guide may also be an incompletely tubular or sheath-like entity, open not only at the proximal and distal ends but also along at least some of its top. In this case, the enclosed guide together with the bottom of the blade comprises the conduit for the passage of the flexible airway instrument. The mechanism for connecting the blade to the enclosed guide depends upon the manifestation of the COID and the materials used to make the blade and the enclosed guide. In FIG. 1, the blade could be a shaped metal bar enclosed in plastic, as described above, while the sheath comprising the enclosed guide could be made of plastic. In this case, the enclosed guide could be attached to the blade by means of, for example, plastic welding or glue.

Depending upon the manifestation of the COID, the enclosed guide may be irreversibly attached to the blade along the full length of both elements, or may be fully or partially removable or detachable from the blade. In addition, depending upon the manifestation of the COID, the enclosed guide may be made of metal, plastic, fabric, or a combination of these materials. In some manifestations of the COID, an endotracheal tube may comprise a part of the enclosed guide.

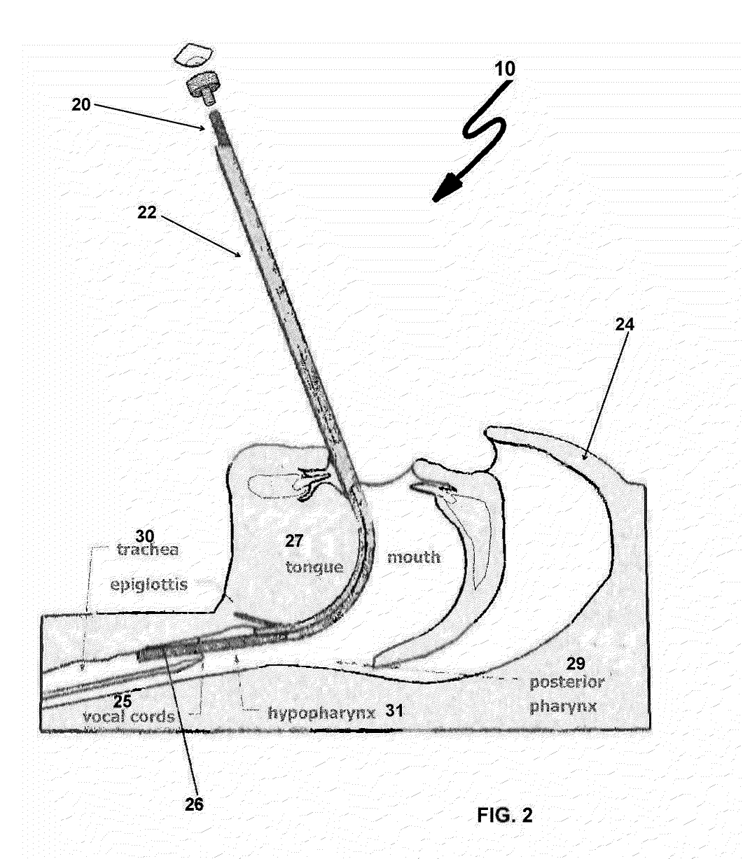

The enclosed guide conforms the fiber optic bronchoscope tightly to the COID, permits the fiber optic bronchoscope to move freely with respect to the COID, and permits the operator to release the fiber optic bronchoscope from the COID. The bronchoscope can be slid into the guide, so that the distal end 26 thereof can come out the distal end 28 of the guide and into the trachea (30, FIG. 2) of the patient 24, as described below with respect to FIG. 2.

The system further comprises a flexible airway instrument 20, in this case, a fiber optic bronchoscope. The bronchoscope comprises a shaft of fiber optics 32 and an eye piece 34 for the operator to look through. The blade further comprises a handle portion 22 to deliver the bronchoscope into the trachea of a patient for co-axial intubation, meaning intubation with, e.g., an endotracheal tube inserted co-axially with the bronchoscope.

As shown in FIG. 2, the blade is long enough that the handle portion remains substantially outside the patient 24 after placement, in order for the operator to manipulate the same. In one example for use with an adult of average size, the blade width is preferably 1 to about 2.5 cms measured straight across, and a length of about 20 to about 30 cms, measured along the “J” curve.

Once the COID has been used to place the distal end of the flexible airway instrument into the trachea, the focus of the present invention, endotracheal intubation with an endotracheal tube can thereafter be accomplished using one of several methods, depending upon the specific characteristics of the COID being used. Each method leads to the same result: an endotracheal tube threaded onto the shaft of the flexible airway instrument and freely moveable along that shaft, permitting the operator to advance the endotracheal tube over the shaft and into the trachea. Each method thus ends with the operator able to complete endotracheal intubation in the standard fashion associated with the use of the fiber optic bronchoscope.

Method 1 of completing endotracheal intubation is used in examples of the COID in which the enclosed guide incorporates a separable endotracheal tube. In this method, the endotracheal tube is already in the patient's mouth, with its distal end close to the vocal cords. The operator releases the endotracheal tube from the COID and advances the endotracheal tube over the bronchoscope and into the trachea. He removes the bronchoscope, leaving the endotracheal tube in the trachea.

Method 2 of completing endotracheal intubation is used in examples of the COID in which the COID can be disassembled in order to release the fiberoptic bronchoscope from the enclosed guide. In this method, an endotracheal tube has been pre-loaded onto the bronchoscope. With the end of the bronchoscope remaining in the trachea, the operator removes the COID from the mouth of the patient and disassembles it, thereby separating it from the shaft of the bronchoscope. After setting the COID aside, the operator distally advances the endotracheal tube over the shaft of the bronchoscope and into the trachea. He removes the bronchoscope, leaving the endotracheal tube in the trachea.

Method 3 of completing endotracheal intubation is used in examples of the COID in which the internal diameter of the enclosed guide is large enough to permit passage of an endotracheal tube. In this method, an endotracheal tube has been preloaded onto the bronchoscope. The operator distally advances the endotracheal tube over the shaft of the bronchoscope, through the COID, and into the trachea. The bronchoscope is then removed, leaving the endotracheal tube in the trachea.

Method 4 is used in examples of the COID in which the internal diameter of the enclosed guide is not large enough to permit passage of an endotracheal tube. This method requires that the shaft of the fiberoptic bronchoscope in use be readily separable from the handle. In this method, the operator separates the shaft of the bronchoscope from its handle. He then removes the COID from the patient's mouth, leaving the distal end of the shaft in the trachea. He slides the COID proximally off the shaft and sets it aside. He places an endotracheal tube onto the proximal end of the shaft and advances the endotracheal tube over the shaft of the bronchoscope and into the trachea. He removes the bronchoscope, leaving the endotracheal tube in the trachea.

The methods of completing intubation described above are merely examples, and are not necessarily mutually exclusive. Other methods of intubation may also be used. However, as noted above, the focus of the present invention is to facilitate placement of the bronchoscope into the trachea.

It should be noted that the examples of the COID in which the enclosed guide incorporates an endotracheal tube are particularly advantageous from the point of view of completing endotracheal intubation. In these examples, the tip of the endotracheal tube is close to the vocal cords, as little as a centimeter away. This has the advantage of permitting the operator to complete the endotracheal intubation by advancing the endotracheal a substantially shorter distance than would otherwise be necessary, and without having to manipulate the endotracheal tube on the proximal shaft of the flexible airway instrument. With these examples, endotracheal intubation should be more readily and directly accomplished.

Insertion of the COID brings the distal end 26 of the fiber optic bronchoscope immediately into the supraglottic space so that the vocal cords 25 can readily be seen through eye piece 34. The COID protects the distal end of the fiber optic bronchoscope from being obscured by soft tissues, especially the tongue 27. The COID also protects the fiber optic bronchoscope from secretions by permitting it to be lifted from the posterior pharynx 29, where secretions tend to collect. The handle portion 22 of the COID permits the operator to easily aim the fiber optic bronchoscope by adjustments in the orientation and placement of the COID. In addition, the COID facilitates the insertion of the distal end of the fiber optic bronchoscope into the trachea 30 by permitting the operator to adjust the aim of the fiber optic bronchoscope as it is advanced.

The COID also allows the operator to be less reliant upon specific skills associated with the use of a fiber optic bronchoscope. The operator does not need to rely as much upon the control mechanisms built into the fiber optic bronchoscope. Instead, the operator is able to rely upon maneuvers similar to those used in traditional direct laryngoscopy, skills that are typically in daily use by the operator.

The system of FIG. 1 is used as follows, described with respect to FIG. 2. The operator chooses a particular implementation of COID, based upon the size of the patient to be intubated and the characteristics of their airway. Except for embodiments where the bronchoscope is integrated with the blade, the operator inserts the distal end of the fiberoptic bronchoscope (or other flexible airway instrument) into the enclosed guide and advances it until the tip of the bronchoscope is close to the end of the blade. He places the distal end 8 of the COID into the mouth of the patient and advances carefully into the hypopharynx 31, monitoring the progress by direct visualization and/or by means of the image obtained through the bronchoscope. With the distal end of the assembly in the hypopharynx, the operator obtains a visual thereof, e.g., by looking into the eyepiece 34 of the fiber optic bronchoscope or at the display (not shown). Using the COID, he alters the orientation and position of the assembly, as necessitated by the presence of secretions and by the physical characteristics of the patient's airway. When the vocal cords 25 come into view, he advances the fiber optic bronchoscope between the vocal cords and into the trachea 30, again using the COID to aim the flexible airway instrument. With the distal end of the bronchoscope in the trachea, the operator completes the endotracheal intubation in one of the ways previously discussed.

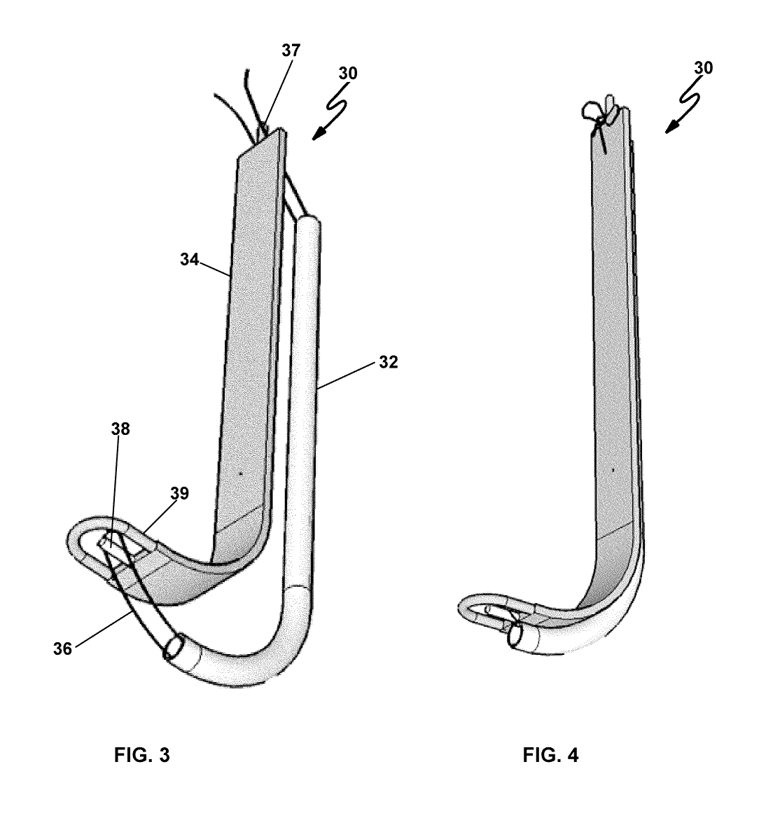

FIG. 3 depicts one example of a COID 30 in accordance with an aspect of the present invention. In this example, the enclosed guide comprises a plastic tube 32 that is separably coupled to J-shaped blade 34 at one or more projecting elements via at least one length of suture, surgical tape or similar material. In one example, a plastic endotracheal tube is used as the enclosed guide. In the present example, there is one length of suture 36 passed through the tube and around projecting elements 37 and 38 at either end of the blade. It is most important from a usage standpoint that the distal end be well anchored, e.g., with a loop, since the distal end is inserted into the patient. At the proximal end, the length of suture could be coupled by looping, clamping, tying (as shown), taped or by other means. Further, the length of suture preferably travels inside the guide to minimize the possibility of it causing any damage to soft pharyngeal tissue. A protective guard 39 is preferably present around projecting element 38. FIG. 4 shows COID 30 from FIG. 3 after being tightly coupled to the blade. If an endotracheal tube is used as the enclosed guide, endotracheal intubation may be completed by Method 1, previously described. The endotracheal tube is separated from the rest of the COID by untying or cutting the suture or tape and withdrawing it from the COID. Otherwise, endotracheal intubation may be completed by Method 4.

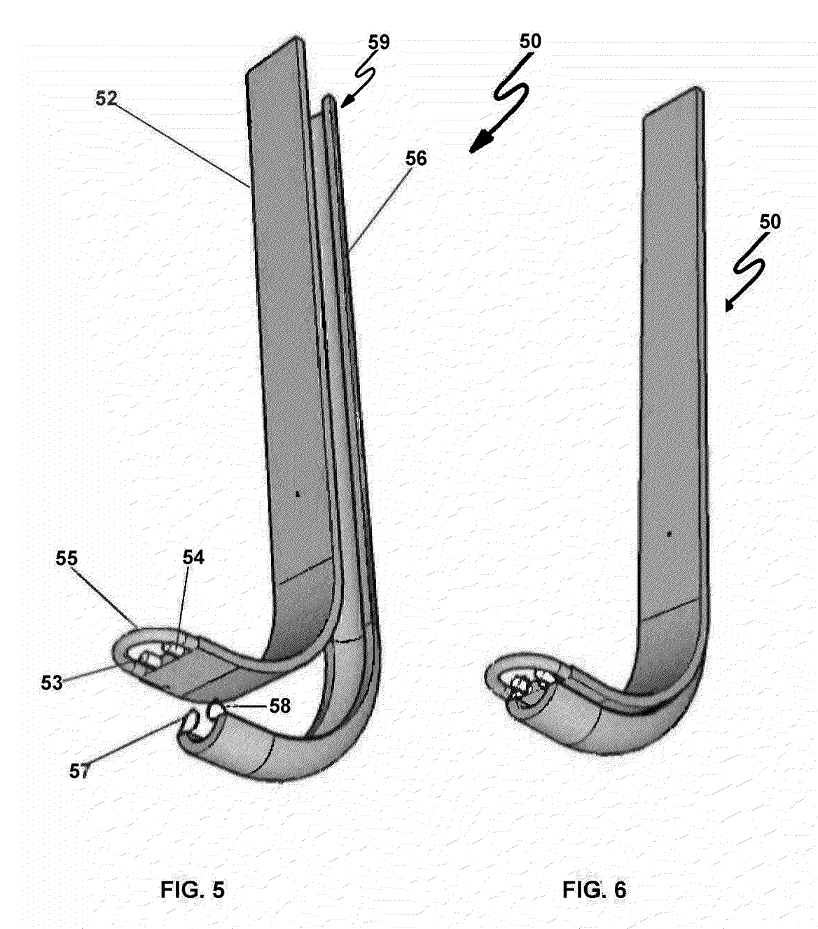

FIG. 5 depicts another example of a COID 50 in accordance with another aspect of the present invention. At a distal end 51 of blade 52 are two projecting elements 53 and 54, surrounded by a protective guard 55. In this example, the enclosed guide takes the form of a semi-tubular structure 56 separably coupled to the blade via two loops 57 and 58 mated with the projecting elements. The enclosed guide is preferably made of an atraumatic material such as soft plastic, with the loops made of a similar material or of embedded suture. The loops are irreversibly attached to the enclosed guide and would be of a similarly atraumatic material or of suture. The proximal end 59 of the semi-tubular structure may be held in place by the operator's hand, clamped, tied or otherwise separably coupled to the blade. The proximal end of the COID is preferably readily separable in order to facilitate decoupling of the COID from the flexible airway instrument. FIG. 6 shows COID 50 after the loops are mated with the projecting elements. While the example in FIGS. 5 and 6 show a COID with two projecting elements mated with two loops, a COID with a different number of projecting elements and loops would also be possible, for example, one projecting element and one loop. Disassembly of this example of the COID is readily accomplished by moving the blade posterior with respect to the enclosed guide, so that the loops disengage from the posts. Endotracheal intubation may be completed by Method 2 described above.

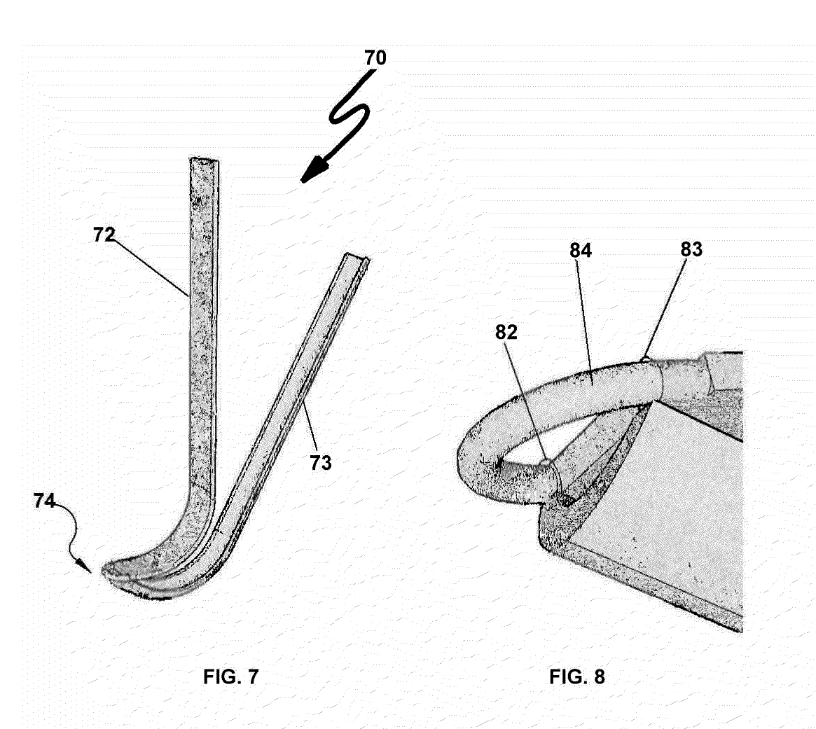

FIG. 7 depicts another example of a COID 70 in accordance with another aspect of the present invention. In this example, the blade 72 and semi-tubular structure 73 are connected at the distal end 74 of the COID. As shown more clearly in FIG. 8, the connection is made by means of loops 82 and 83 encircling U-shaped extension 84 at the distal end of the blade. Alternatively, the connection could be, for example, by means of paired hinge-like structures. The loops are irreversibly attached to the enclosed guide and made of plastic or suture or other atraumatic material, preferably elastic in nature. As with the prior example, the proximal end of the semi-tubular structure may be held in place by the operator's hand, clamped, tied or otherwise separably coupled to the blade. Endotracheal intubation may be completed by Method 4.

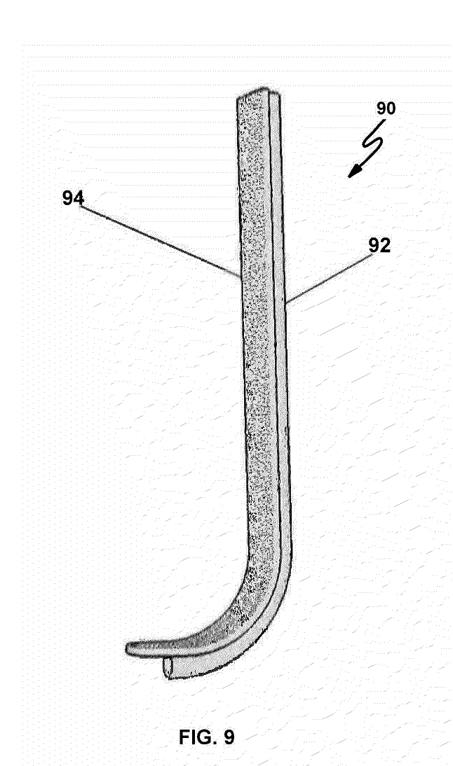

FIG. 9 depicts still another example of a COID 90 in accordance with another aspect of the present invention. In this example, the enclosed guide takes the form of a tubular structure 92 connected to blade 94. The example of FIG. 9 could be constructed entirely of metal, with the enclosed guide welded to the blade. Alternatively, the blade could be formed of plastic or of metal and plastic or of metal and fabric, and the tubular structure could be plastic or of fabric, as previously described, in which case the enclosed guide would be connected to the blade by means of a non-toxic glue or by the process of plastic welding. Endotracheal intubation may be completed by Method 4.

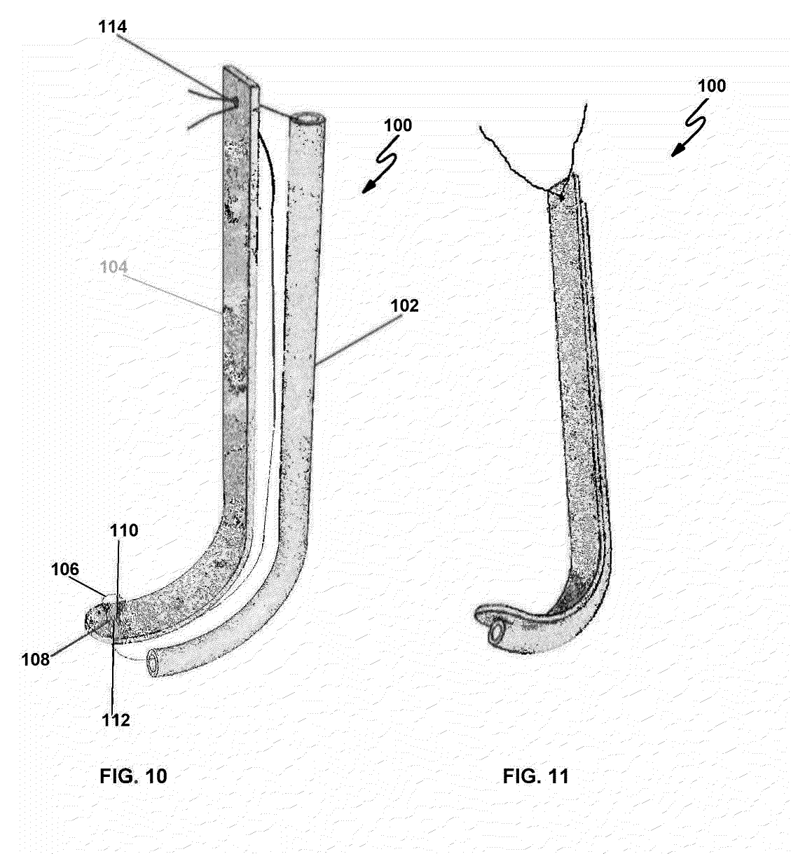

FIG. 10 depicts another example of a COID 100 in accordance with yet another aspect of the present invention. In this example, the enclosed guide comprises a plastic tube 102 that is separably coupled to J-shaped blade 104 at one or more blade openings via at least one length of suture or surgical tape. In the present example, there is one length of suture 106 passed longitudinally through the tube, through each of distal openings 108 and 110 and around separator 112 situated between openings 108 and 110, and finally through opening 114 at the proximal end of the blade. FIG. 11 shows COID 100 from FIG. 10 after being tightly coupled to the blade by tightening the length of suture. The endotracheal tube is separated from the rest of the COID by untying or cutting the suture or tape and withdrawing it from the COID. Endotracheal intubation may be completed by Method 1 described above.

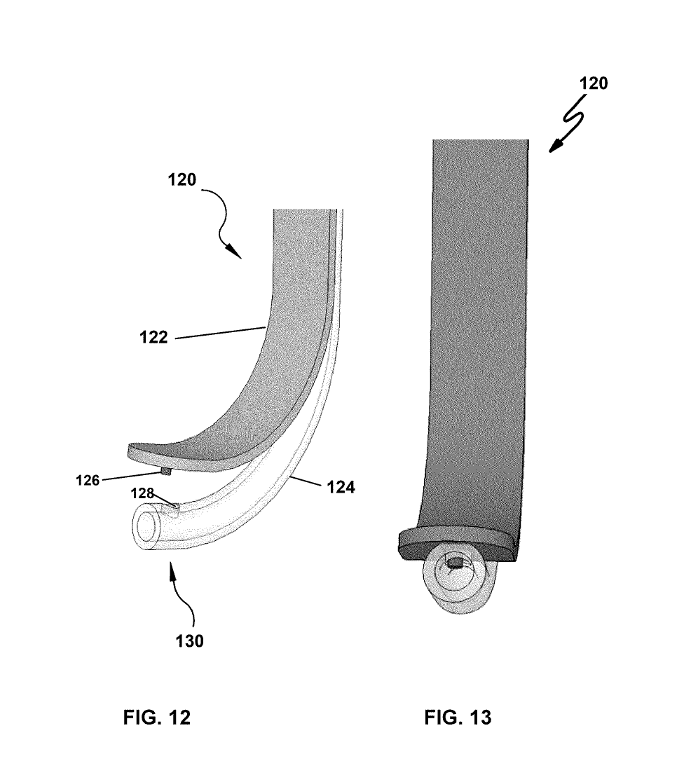

Depicted in FIG. 12 is another example of a COID 120 in accordance with another aspect of the present invention. The COID comprises J-shaped blade 122 and endotracheal or similar plastic tube 124, separably coupled at the distal end 130 via post (126) and hole (128) connection. The operator forms the connection by placing the post into the hole and by putting tension on the tube in the proximal direction. FIG. 13 shows COID 120 from FIG. 12 after the tube is tightly coupled to the blade via the post-and-hole connection. The endotracheal tube is separated from the rest of the COID by releasing the tension on the tube, allowing the hole to decouple from the post. Endotracheal intubation may be completed by Method 1.

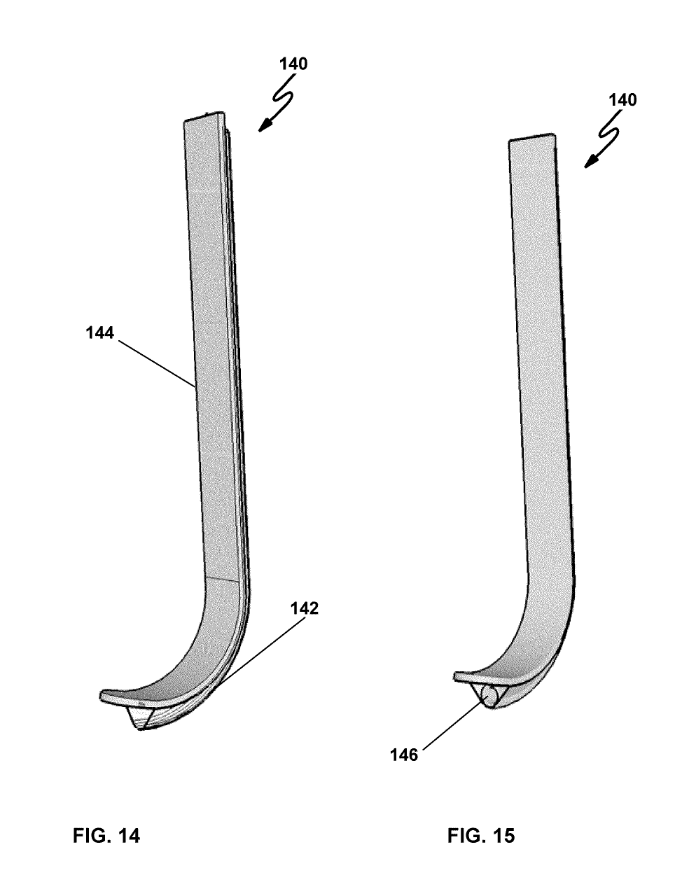

FIG. 14 depicts another example of a COID 140 in accordance with another aspect of the present invention. In this example, the enclosed guide takes the form of a flexible sheath 142 open at both ends and longitudinally attached to the back of J-shaped blade 144. The flexible sheath may be made of, for example, plastic or of polyester mesh or fabric. Depending upon the materials comprising the blade, the flexible sheath may be attached to the blade by means of, for example, glue or plastic welding. Preferably, the flexible sheath is supple, like fabric, or baggy or a somewhat heavier plastic sheet, with internal dimensions sufficient to permit passage of an endotracheal tube. Optionally, as shown in FIG. 15, an endotracheal tube 146 may be pre-loaded in the sheath. Endotracheal intubation may be completed by Method 3.

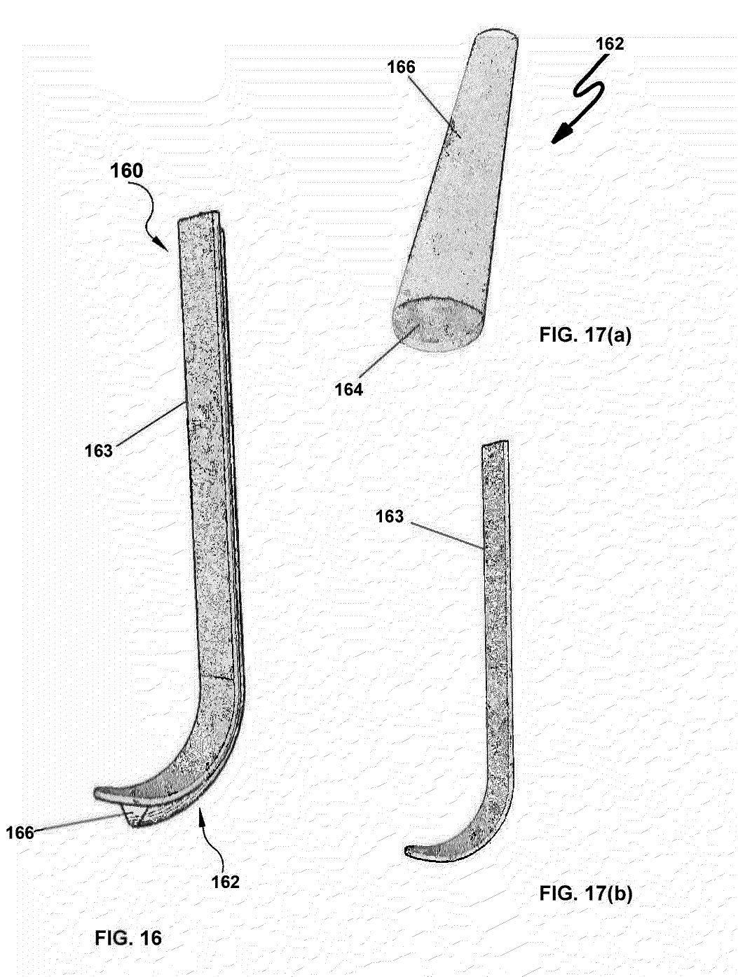

Depicted in FIGS. 16, 17(a) and 17(b) is another example of a COID 160 in accordance with another aspect of the present invention. In this example, the enclosed guide takes the form of a flexible sheath 162 (FIG. 17(a)) coupled to J-shaped blade 163 (FIG. 17(b)) and having two portions 164 and 166. The sheath can be made of a flexible material, such as, for example, plastic or polyester mesh or fabric or other medical fabric. Portion 164 is open proximally and closed distally, having dimensions similar to those of the blade. The second portion 166 is open at both ends. With the blade inserted into the first portion of the sheath, the second portion of the sheath conforms to the shape of the blade and comprises the enclosed guide. Preferably, the second portion of the sheath has internal dimensions sufficient to permit passage of an endotracheal tube. Endotracheal intubation may be completed by Method 3.

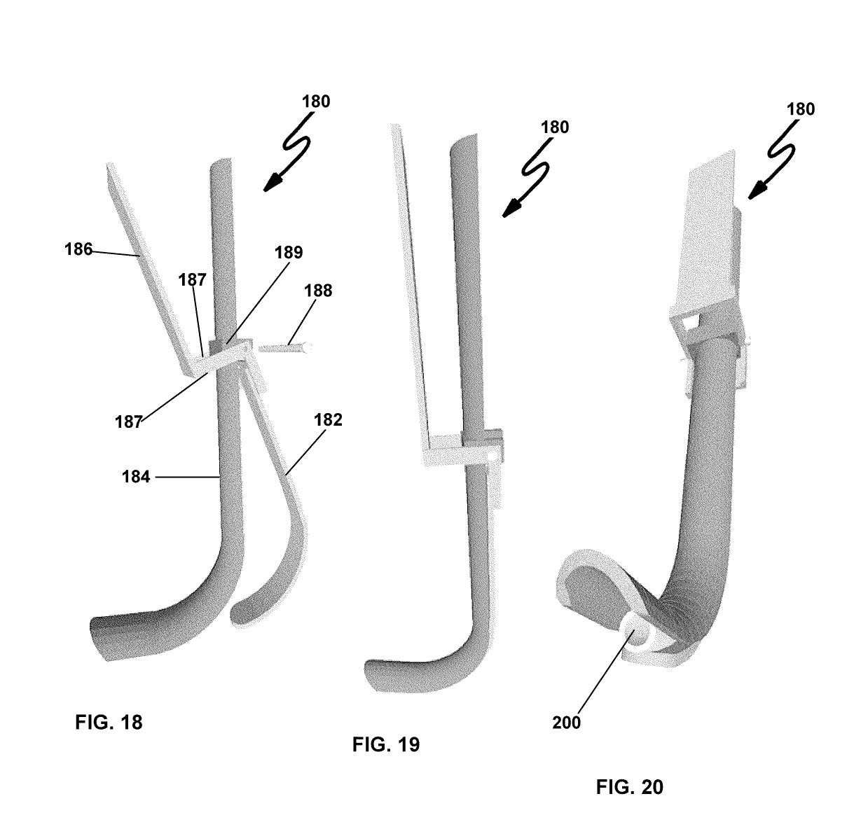

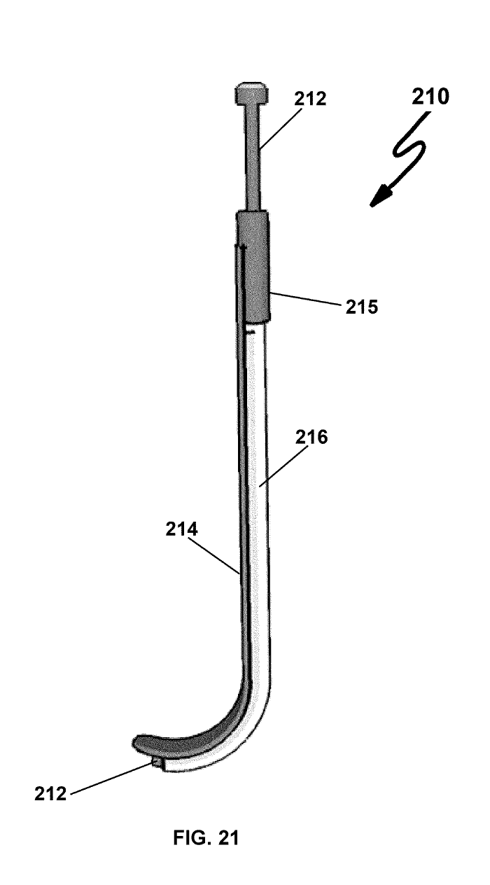

FIG. 18 depicts another example of a COID 180 in accordance with still another aspect of the present invention. In this example, the enclosed guide takes the form of a secondary J-shaped blade 182 that is pivotally coupled to the main J-shaped blade 184. The secondary blade comprises a handle 186 offset from blade 182 via arms 187. The mechanism for pivotal coupling can take many forms, in this example via pin 188 through fixed member 189 attached to the main blade. FIG. 19 shows the secondary blade in the closed position relative to the main blade. As shown best in FIG. 20, the secondary blade is sized and shaped to create an opening with the main blade when closed by the handle. In this example, the primary blade is concave, while the secondary blade is straight. In this example, in order to accomplish endotracheal intubation, the COID is removed from the mouth and disassembled by removal of the pin 188, permitting endotracheal intubation Method 4 to be used. Alternatively, as shown in FIG. 20, an endotracheal or similar plastic tube 200 may be pre-loaded between the blades. In this case, the endotracheal tube is released by diminishing the tension on the blades, permitting endotracheal intubation Method 1 to be used.

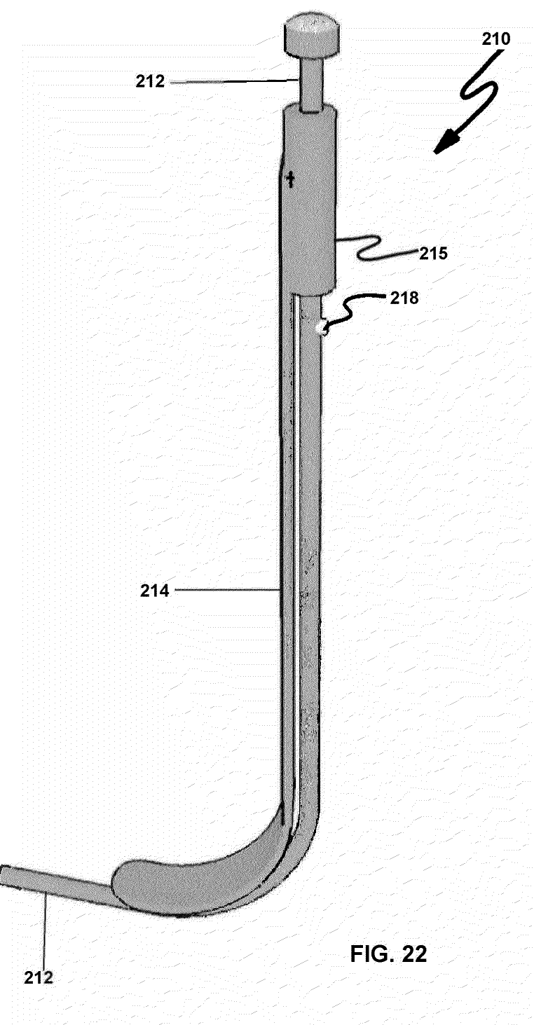

Depicted in FIG. 21 and FIG. 22 is yet another example of a COID 210 in accordance with another aspect of the present invention. In this example, a fiber optic bronchoscope or other flexible airway instrument 212 is attached to J-shaped blade 214 via a hollow cylindrical member 215 fixed to the blade. The hollow cylindrical member has an internal diameter slightly larger than that of the shaft of the bronchoscope so that the bronchoscope moves freely inside this member. The enclosed guide 216 in this example comprises the second portion of a two-portion sheath, as described with respect to FIGS. 16, 17(a) and 17(b), the first portion of the sheath fitting around the blade. The bronchoscope operates similar to that described with respect to FIG. 1. An endotracheal tube (not shown) is pre-loaded in the enclosed guide such that the shaft portion of the bronchoscope fits inside the tube. The dimensions of the enclosed guide are sufficient for the endotracheal tube to move freely within it. FIG. 22 shows the blade/bronchoscope assembly without the enclosed guide, with the fiber optic bronchoscope 212 partially advanced. A stop 218 is attached to the shaft of the bronchoscope, effectively attaching it to the COID 217 by limiting its proximal movement. The operator separates the endotracheal tube from the enclosed guide by sliding it distally out of the enclosed guide, permitting endotracheal intubation Method 1, described above, to be used.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. An co-axial oral intubation device, comprising:
a generally J-shaped blade with a handle portion to deliver a flexible airway instrument when coupled to the blade into a trachea of a patient for subsequent co-axial intubation with the flexible airway instrument, wherein the blade is long enough that the handle portion remains substantially outside the patient after placement in the patient; and
an enclosed guide coupled to a back side of the blade for guiding and holding the flexible airway instrument when present, wherein the guide conforms to the blade.

2. The co-axial oral intubation device of claim 1, wherein the guide is integral with the blade.

3. The co-axial oral intubation device of claim 1, wherein the guide is separably coupled to the blade.

4. The co-axial oral intubation device of claim 3, wherein the guide is separably coupled to the blade via suture or cloth tape looped through a plurality of holes in the blade such that the guide is tightly coupled to the blade along a length of the guide.

5. The co-axial oral intubation device of claim 3, wherein the guide and blade are separably coupled via at least one post-and-hole connection.

6. The co-axial oral intubation device of claim 3, wherein another blade is pivotally coupled to the blade, wherein the another blade is lockable in place against the back side of the blade, and wherein the guide is held between the blades.

7. The co-axial oral intubation device of claim 3, wherein the blade comprises at least one projecting element at a distal end of the blade, and wherein the guide is separably and tightly coupled to the blade via suture or cloth tape at the at least one projecting element.

8. The co-axial oral intubation device of claim 7, wherein the guide comprises an opening at a distal end thereof, and wherein the suture or cloth tape is passed through the opening and looped around the at least one projecting element.

9. The co-axial oral intubation device of claim 3, wherein the guide comprises an endotracheal tube.

10. The co-axial oral intubation device of claim 1, wherein the guide comprises a first section and a second section, and wherein one of the first section and the second section fits within the other of the first section and the second section when brought together.

11. The co-axial oral intubation device of claim 10, wherein the guide is separably coupled to the blade via a flexible or semi-rigid sheath coupled longitudinally to a front top of the guide, and wherein the blade is inserted into the sheath to conform the guide tightly to the blade.

12. The co-axial oral intubation device of claim 1, wherein the guide is attached to the blade.

13. The co-axial oral intubation device of claim 12, wherein the guide comprises an endotracheal tube.

14. The co-axial oral intubation device of claim 1, wherein the guide is tubular in shape.

15. The co-axial oral intubation device of claim 1, wherein the guide is semi-tubular in shape.

16. The co-axial oral intubation device of claim 15, wherein one of the guide and the blade comprises a plurality of female elements at the distal end thereof, wherein the other of the guide and the blade comprises a plurality of male elements at a distal end thereof, sized to fit within the female elements, and wherein the guide couples to the blade by fitting together the plurality of female elements and the plurality of male elements.

17. The co-axial oral intubation device of claim 15, wherein the guide is fixed to the blade at a distal end thereof.

18. The co-axial oral intubation device of claim 1, wherein the guide is flexible.

19. The co-axial oral intubation device of claim 1, wherein the guide is semi-rigid.

20. The co-axial oral intubation device of claim 1, wherein the blade is rigid.

21. The co-axial oral intubation device of claim 1, wherein the blade is malleable.

22. The co-axial oral intubation device of claim 1, wherein the guide is rigid.

23. The co-axial oral intubation device of claim 22, wherein the flexible airway instrument is integral with the guide.

24. A system for oral intubation, comprising:
a flexible airway instrument; and
a co-axial oral intubation device, the co-axial oral intubation device comprising:
a generally J-shaped flat blade with handle portion to deliver the flexible airway instrument when coupled to the blade into a trachea of a patient for subsequent co-axial intubation with the flexible airway instrument, wherein the blade is long enough that the handle portion remains substantially outside the patient after placement in the patient; and
an enclosed guide coupled to a back side of the blade for guiding and holding the flexible airway instrument, wherein the guide conforms to the blade.

25. The system of claim 24, wherein the flexible airway instrument comprises a fiber optic airway instrument.

26. The system of claim 24, wherein the flexible airway instrument comprises a laryngoscope.

27. The system of claim 24, wherein the flexible airway instrument comprises a bronchoscope.

28. The system of claim 24, wherein the flexible airway instrument comprises a videoscopic airway device.

29. The system of claim 24, further comprising a tracheal tube.

30. The system of claim 24, wherein the guide is integrated with the blade.

31. The system of claim 30, further comprising a tracheal tube.

32. The system of claim 24, wherein the guide is integral with the blade.

33. The system of claim 24, wherein the guide is separably coupled to the blade.

34. The co-axial oral intubation device of claim 33, wherein the guide comprises an endotracheal tube.

35. The system of claim 33, wherein the guide is separably coupled to the blade via suture or cloth tape looped through a plurality of holes in the blade such that the guide is tightly coupled to the blade along a length of the guide.

36. The system of claim 33, wherein the guide and blade are separably coupled via at least one post-and-hole connection.

37. The system of claim 33, wherein another blade is pivotally coupled to the blade, wherein the another blade is lockable in place against the back side of the blade, and wherein the guide is held between the blades.

38. The system of claim 33, wherein the blade comprises at least one projecting element at a distal end of the blade, and wherein the guide is separably and tightly coupled to the blade via suture or cloth tape at the at least one projecting element.

39. The system of claim 38, wherein the guide comprises a lumen, and wherein the suture or cloth tape is passed through the lumen and looped around the at least one projecting element.

40. The system of claim 33, wherein the guide is separably coupled to the blade via a flexible or semi-rigid sheath coupled longitudinally to a front of the guide, and wherein the blade is inserted into the sheath to conform the guide tightly to the blade.

41. The system of claim 24, wherein the guide is attached to the blade.

42. The co-axial oral intubation device of claim 41, wherein the guide comprises an endotracheal tube.

43. The system of claim 24, wherein the guide is tubular in shape.

44. The system of claim 24, wherein the guide is semi-tubular in shape.

45. The system of claim 44, wherein one of the guide and the blade comprises a plurality of female elements at the distal end thereof, wherein the other of the guide and the blade comprises a plurality of male elements at a distal end thereof, sized to fit within the female elements, and wherein the guide couples to the blade by fitting together the plurality of female elements and the plurality of male elements.

46. The system of claim 44, wherein the guide is fixed to the blade at a distal end thereof.

47. The system of claim 24, wherein the guide is flexible.

48. The system of claim 24, wherein the guide is semi-rigid.

49. The system of claim 24, wherein the blade is rigid.

50. The system of claim 24, wherein the blade is malleable.

51. The system of claim 24, wherein the guide comprises a first section and a second section, and wherein one of the first section and the second section fits within the other of the first section and the second section when brought together.

52. The system of claim 24, wherein the guide is rigid.

53. The system of claim 52, wherein the flexible airway instrument is integral with the guide.
